(19) United States
(12) Patent Application Publication
(10) Pub. No.: US 2004/0199135 A1
(43) Pub. Date: Oct. 7, 2004

(54) Disposable hygienic absorbent for geriartic use for men

(76) Inventor: Herbert Engler, Belo Horizonte (BR)

Correspondence Address: Gottlieb Rackman & Reisman, 270 Madison Avenue, New York, NY, 10016, US

(21) Appl. No.: 10/476,555

(22) PCT Filed: Apr. 30, 2002
(86) PCT No.: PCT/BR02/00061

(30) Foreign Application Priority Data
Apr. 30, 2001 (BR) MU8101024-9

(51) Int. Cl. A61F 13/15; A61F 13/20

(52) U.S. Cl. 604/385.01; 604/386

(57) ABSTRACT

The present invention relates to a new hygienic absorbent for geriatric use and for use in hospitals and the like, more specifically for use by male patient suffering from urinary incontinence, that is, that have problems in retaining the urine, even if only occasionally. The problem faced and solved by the present invention applies mainly to the geriatric market and to post-surgery care of prostate and urinary apparatus and the present invention refers to an hygienic absorbent particularly designed to avoid that the clothes used be moistened by contact with the body secretions. The disposable hygienic absorbent for geriatric use for men according to the present invention comprises a small, anatomically conformed body (1) manufactured from a material having the property of absorbing liquids, said body (1) having a substantially trapezoidal configuration with a smaller base (2) having approximately half the length of the upper base (3) and slightly curved lateral sides (4), with free peripherical edges (5) and a central portion (6) with suitable dimensions for receiving a layer of an adhesive material and a protective peel-off cover (7) applied over said central portion (6).

### BACKGROUND OF THE INVENTION

1. Field of the Invention

The present invention relates to a new hygienic absorbent for geriatric use and for use in hospitals and the like. More specifically, the present invention refers to an hygienic absorbent particularly designed for use by male patient suffering from urinary incontinence, that is, that have problems in retaining the urine.

2. Description of the Prior Art

As it is widely known, after a certain age it is usual for men to present a tendency to have prostate problems that in most cases need a surgical procedure to be solved.

Usually, after the required hospital and medical care the patient is perfectly able to return to his normal day to day life activities. However, this type of surgery leaves a permanent damage that most known surgical techniques cannot avoid, which is the urinary incontinence.

Several researches were made and it was observed that this is a market niche still unexplored and that there are no really efficient devices that are also comfortable designed for daily use. What can be found on the market are adaptations and variations of the disposable diapers originally designed for children and redesigned for adult sizes, mainly destined for patients in bed, that require products with a great capacity of absorbing liquids.

Outside this segment, there were found no devices with small dimensions, for daily use, similar to the disposable, hygienic absorbents for women, which are small and practical.

The problem faced and solved by the present invention applies mainly to the geriatric market and to post-surgery care of prostate and urinary apparatus and the present invention refers to an hygienic absorbent particularly designed to avoid that the clothes used be moistened by contact with the body secretions.

### SUMMARY OF THE INVENTION

With the above problems in mind, a product having small dimensions, anatomically tested and found to be extremely comfortable was developed, which has the capability of absorbing small amounts of bodily secretions, namely urine, similarly to a modern feminine hygienic absorbent.

Additional objects and advantages of the invention are set forth in part in the description which follows and in part are obvious from the description, or may be learned from the practice of the invention. The objects and advantages of the invention also may be realized and attained by means of the features particularly pointed out in the attached claims.

### BRIEF DESCRIPTION OF THE DRAWINGS

The accompanying drawings, which are incorporated in and constitute a part of the specification, illustrate a preferred embodiment of the invention, and together with the description serve to explain the principles of the invention. In the drawings:

FIGS. 1 to 6 are different perspective views of the disposable absorbent applied to a piece of underwear;

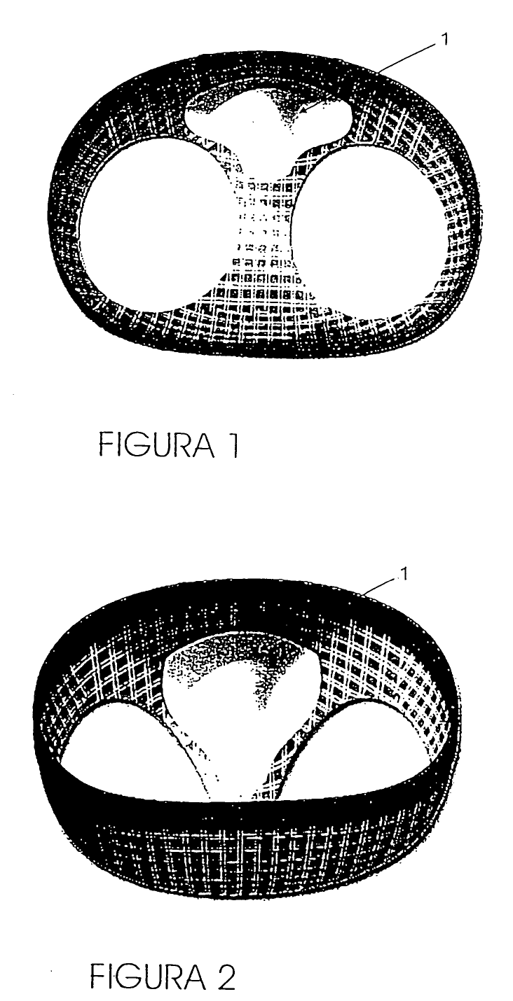

FIG. 6 is a schematic representation of the disposable absorbent with the protective peel-off cover partially removed;

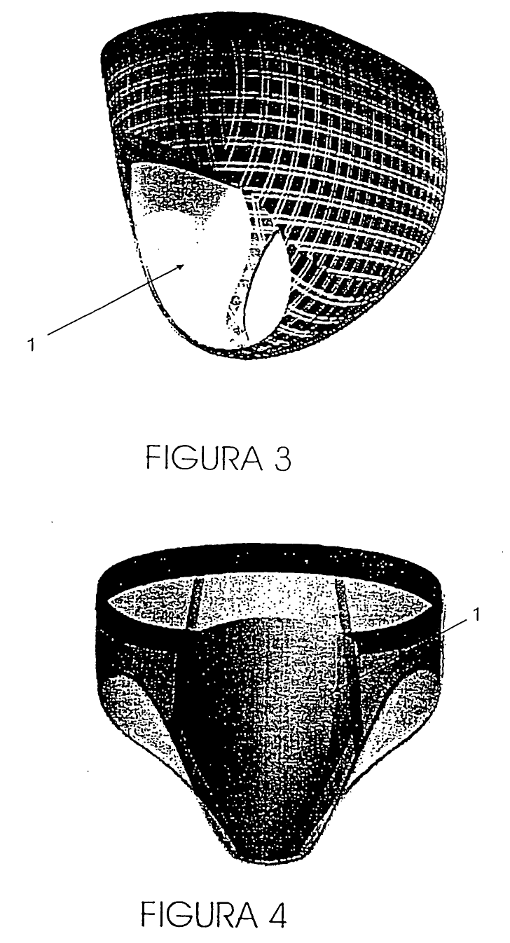

FIG. 7 is a perspective view of the disposable absorbent ready to be positioned within the underwear; and

FIG. 9 is a side elevational view showing the positioning of the disposable absorbent in the underwear.

### DESCRIPTION OF A PREFERRED EMBODIMENT

A specific embodiment of the presently preferred embodiment of the present invention is illustrated as example in the attached drawings and will be further described in detail hereinafter. However, it shall be understood that while the present invention is susceptible to several modifications and alterations of configuration and dimensions, the purpose of the present specification is not to limit the invention to the particular forms and/or dimensions described herein but, instead, to cover all the modifications and alternative embodiments that are within the spirit and scope of the invention, as defined by the attached claims.

With particular reference now to the drawings, the object of the present invention is a disposable hygienic absorbent particularly designed for use by male patient suffering from urinary incontinence. The absorbent 1 essentially comprises a small, anatomically conformed body, manufactured from a material having the property of absorbing liquids.

The body portion 1 has a substantially trapezoidal configuration with a smaller base 2 having approximately half the length of the upper base 3, and its lateral sides 4 are slightly curved, with free peripherical edges 5 to provide additional comfort, said body 1 providing an anatomically comfortable format for receiving and accommodating the penis of the user.

Said body 1 has a central portion 6 with suitable dimensions for receiving a layer of an adhesive material and a protective peel-off cover 7 is applied over said central portion 6.

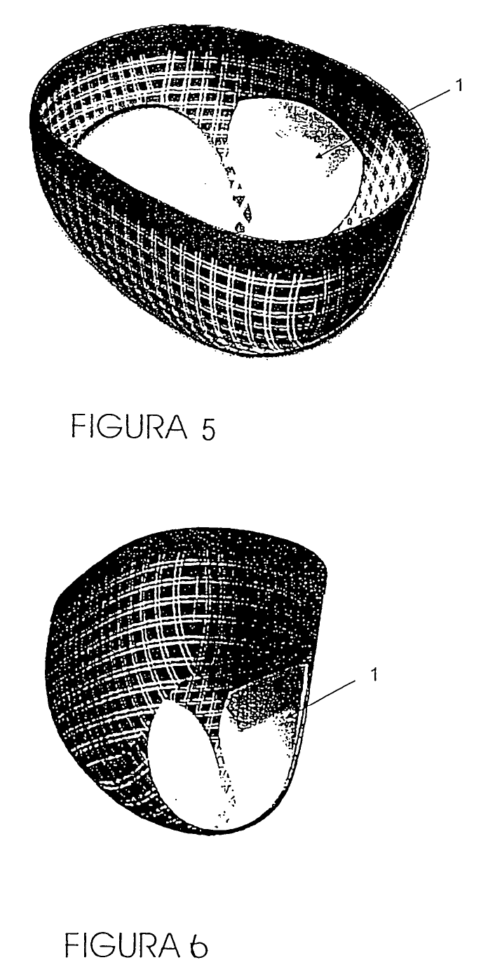

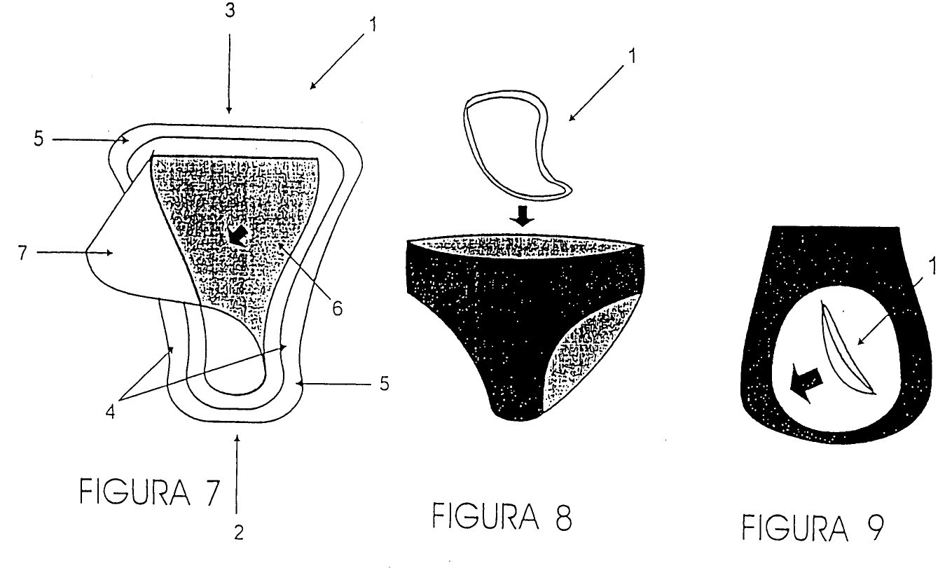

FIG. 7 shows the disposable hygienic absorbent with the peel-off protective cover 7 partially removed and FIGS. 8 and 9 show the manner and position said absorbent body 1 is positioned within a piece of underwear.

The absorbent material to be used can be a filler comprised of a thin layer of cellulose impregnated with an absorbing product.

Having described and illustrated what is considered the best form presently contemplated for embodying the present invention, various modifications e variations in its form of manufacture will be promptly apparent to those skilled in the art. Therefore, it is to be understood that the present invention is not limited to the practical aspects of the actual preferred embodiments hereby illustrated and described, and that any such modifications and variations must be considered as being within the spirit and the scope of the invention, as described in the attached claims.

What is claimed is:

1. A disposable hygienic absorbent for geriatric use for men, said absorbent essentially comprising a small, anatomically conformed body (1) manufactured from a material having the property of absorbing liquids, said body (1) having a substantially trapezoidal configuration with a smaller base (2) having approximately half the length of the upper base (3) and slightly curved lateral sides (4), with free peripherical edges (5) and a central portion (6) with suitable dimensions for receiving a layer of an adhesive material and a protective peel-off cover (7) applied over said central portion (6).
